(19) United States
(12) Patent Application Publication
(10) Pub. No.: US 2002/0198507 A1
(43) Pub. Date: Dec. 26, 2002

(54) An Evacuation pouch

(76) Inventor: Jo Ann Ferguson McCorvey, Houston, TX (US)

Correspondence Address: WILLIAM E. JOHNSON,JR, THE MATTHEWS FIRM, 1900 WEST LOOP SOUTH,SUITE 1800, HOUSTON,, TX, 77027, US

(21) Appl. No.: 09/892,393

(22) Filed: Jun. 26, 2001

(51) Int. Cl. A61M 1/00; A61F 5/44

(52) U.S. Cl. 604/355; 604/317; 604/327

(57) ABSTRACT

The e.v. pouch is a latex bag with a structured and contoured adhesive opening designed to fit securely around the outer skin of the rectum. This allows defecation to occur into the pouch. Its main use is for the bowel incontinent patient. It provides a convenient yet sanitary means of disposing of feces with very little exposure of it to the skin. It should dramatically decrease the number of bedsores and skin irritation in the incontinent patient.

### BACKGROUND OF THE INVENTION

The evacuation pouch is a pouch which is used medically in patients who have bowel incontinance. Many patients in hospitals become so sick and feeble that they become incontinent as to bowel and/or bladder. In hospital settings, a urinary catheter is often used to help a patient void urine and also as a way of preventing them from continuously wetting their clothing. However, for G.I. incontinence there is really not a state of the art, except adult diapers and disposable pads to be placed underneath the patient, or a bed pan. When the patient does have a bowel movement with the diaper or pad, there is extensive cleaning that must take place to render the patient free of fecal material. On the other hand, the bed-pan requires extensive preparation and a patient who is aware enough to know in advance that it is needed. This takes up the nurses valuable time in getting the bed-pan, adjusting it, removing it, cleaning up the patient and often having to get it again in 10 minutes. Many patients who need to use a bed pan complain that it is uncomfortable and difficult. Some are so sick that their bodies can not be put in the position to use a bedpan due to pain and/or discomfort or paralysis.

The evacuation pouch (e.v. pouch) provides a convenient and sanitary method of addressing the problem of bowel incontinence. It can be attached to the body and left in place until evacuation occurs. This takes the fecal material away from the body and into the expanding pouch which can then be carefully removed and disposed off with out having an extensive clean up to do.

Some patients are so debilitated that defecation is a semi-continuous occurrence. The use of the e.v. pouch not only keeps the patient clean and irritation free, but it also allows more effective utilization of the nursing staff. In addition, it minimizes nursing staffs from contact with pathogens present in feces. Also, with proper training, the e.v. pouch can be used by ambulatory patients who are home bound because of bowel incontinence.

### BRIEF SUMMARY OF EVACUATION POUCH

The evacuation pouch or e.v. pouch is to be used on bowel incontinent patients, especially, in a hospital setting or extended care facilities where skilled attention and care in the application, care and removal of the pouch will occur. Special care will be needed to prevent any breakage of the delicate and often thin skin around the outside of the rectum. The pouch is more sanitary than diapers in the bed-ridden patients. The e.v. pouch minimizes exposure and contact of the feces to the outer skin.

With proper application, the pouch can be used on ambulatory patients who are incontinent. However, the most effective use will be in an inclined position. The pouch can also be used in a hospital setting when entire fecal expulsion needs to be retrieved for the lab or measured. The pouch should be large and expandable enough so that it will easily hold one or more entire bowel movements. The ideal and most sanitary use would be one complete bowel movement, but with the capability and strength of the material to hold more than one, for convenience sake. The e.v. pouch will be packaged in a sterile container with the appropriate cleaning apparatus to be used prior to attachment of the adhesive mouth of the e.v. pouch to the skin surrounding the rectum.

### BRIEF DESCRIPTION OF THE SEVERAL VIEWS OF THE DRAWING

FIG. 1 shows a side view of the e.v. pouch with the e.v. pouch attached to the body. The body of the e.v. pouch will lay flat against the bed and/or body. FIG. 1A shows the re-inforced bottom of the e.v. pouch which is a thicker layer that helps the e.v. pouch lay flat. FIG. 1A shows a side view of the reinforced end which lays flat against the bed and/or body. FIG. 1B shows the elongated striations of the e.v. pouch which helps in the expansion of the bag while still allowing for a rather compact size.

FIG. 2 shows the details of how the bag will look when first placed on the body. FIG. 2E shows the direction of the adhesive mouth flat against the body with 2E towards the front of the rectum. FIG. 3 shows how the e.v. pouch will expand when viewed from a top. Again, FIG. 2C shows the striations of the e.v. pouch prior to elimination. FIG. 2A shows the thicker end of the e.v. pouch which due to its weight will help the pull of gravity to cause the fecal material to flow down into the bottom of the pouch, away from the body. It also serves as a stabilizer as well as re-inforcement when the pouch is removed and held up right to be disposed of.

FIGS. 4, 5, 6, & 7 shows the adhesive lined (very soft and pliable) mouth opening of the e.v. pouch and the four shapes from a front view. It fits against and around the outer part of the rectum. The mouth of the e.v. pouch fits circular around its top and extends outward with the rim of the mouth extending beyond the circumference of the neck. When the mouth of the e.v. pouch is applied properly it opens up the e.v. pouch so defecation can easily occur into the pouch. The neck, FIG. 2D of the pouch, is circular and narrower than the rest of the pouch. This will aid the movement of the fecal material away from the body. While FIG. 3F shows how the rim of the mouth extends outward in width than the neck of the e.v. pouch.

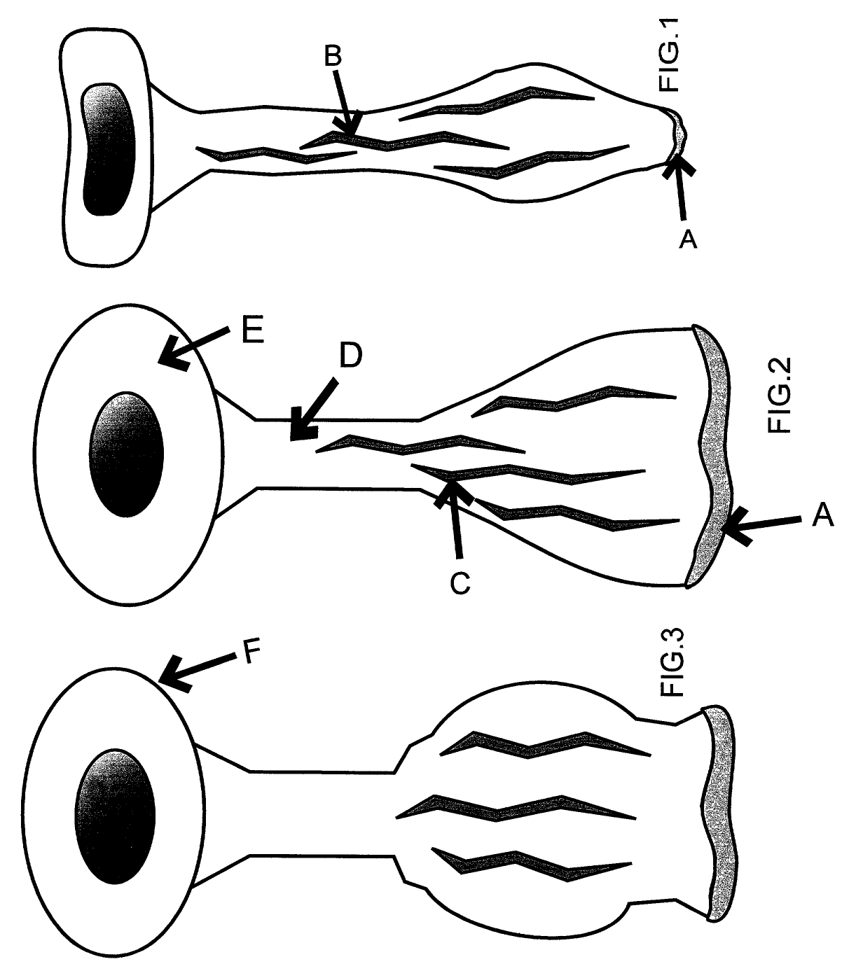

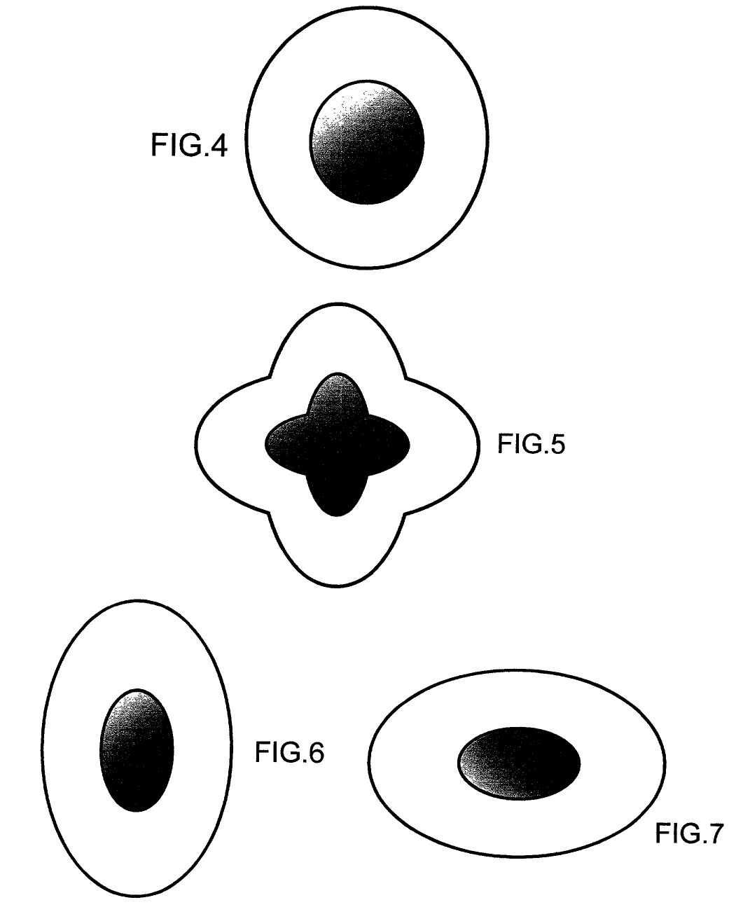

### DETAILED DESCRIPTION OF THE INVENTION

The e.v. pouch is made of a pliable, but strong latex. The material is similar to the material used on diaphragms but much softer. Latex is light weight but strong. The striations of the material will keep the size small yet with the ability to expand as it is used. The shape of the e.v. pouch embodies its usefulness and uniqueness. Each aspect of the design is important as to its overall function. The mouth of the e.v. pouch has four uniquely different shapes to embody the variations in individual body shapes. The different shapes of the outer adhesive mouths are: 1) round; 2) oval; 3) what can be described as the butterfly or four sided club; and 4) rectanglular. However, the opening inside of the mouth will always be circular. The variations in the shape are apart of the patent, and necessary to make the e.v. pouch functional. However, each variation of the mouth will be folded indicating the proper direction for application to the body. The mouth of the e.v. pouch consists of a flat surface with an adhesive strip on the inner side which when applied to the body lays flat against the skin. The e.v. pouch is anchored to the body by the adhesive strip. This adhesive mouth extends outward beyond the opening of the neck of the e.v. pouch forming a wide rim. The width of the mouth's rim depends on the size and shape, ranging from ¾ up to 5 to 6 inches. The material used for the adhesive is similar to the consistency of adhesive tape. The adhesive flat mouth will secure the e.v. pouch to the body, but also it will be pliable enough to remove the e.v. pouch from the skin without abrasions.

The adhesive strip mouth fits perpendicular to the body of the e.v. pouch. There is no prior art as to this type of anchoring of a pouch to the body and in particular for this purpose. Additionally, there is little prior art as to the shape of this device. If we consider the condom, the women's condom or the diaphragm, the shape of the e.v. pouch is completely different. The contoured shape of the e.v. pouch is uniquely designed. It is designed not as a barrier, but as a facilitator of the movement of the fecal material away from the body. In addition to the uniqueness of the shapes of the mouth of the e.v. pouch, it will come in various sizes of the width and shapes of the mouth. This is necessary not only for perfect fit, but also to change the pressure points of the adhesive to the skin. This will allow the skin around the rectum to air on alternate days. This will cut down on the potential abrasion of the skin. Also, the e.v. pouch will come in various sizes according to the size of the individual (XS, S, M, L, XL, 2XL, 3XL, etc.) In this way a person's shape and body build can be allowed for. These sizes will not only encompass the size of the pouch portion, but will also have a different size mouth openings. This will allow for the most accurate fit for the person as well as comfort and proper utility. The shape of the e.v. pouch as stated earlier, is a very important aspect. When the e.v. pouch is applied properly, with front and back in its proper position on the body, the e.v. pouch will lay flat on the bed extending from under the buttocks to the portion between the upper thighs. Additionally, a patient can be placed on their side for convenience or comfort. When this position is used, the e.v. pouch must still be applied correctly, but care must be taken to make sure the bottom of the e.v. pouch rests on the bed or other solid prop as needed. However, if worn under underwear, they must be loose fitting so they will not interfere with the expansion of the e.v. pouch. The portion of the e.v. pouch that will assure its flat position is the shape of the bottom of the e.v. pouch. The bottom of the e.v. pouch will consist on a heavier reinforced flat edge. It will sturdy the e.v. pouch and cause it to lay flat. It will also give the e.v. pouch the re-inforcement it needs when the pouch is removed from the body for disposal. The pouch can be re-sealed and disposed of, or can be tied off for disposal. The e.v. pouch opens when the mouth of the e.v. pouch is attached to the body.

The neck of the pouch is narrower than the bottom. This shape helps to facilitate the fecal materials movement to the bottom portion of the e.v, pouch. The striation of the latex is an added feature in the movement of the fecal material to t he bottom of the e.v. pouch. These striations also facilitate additional expansion of the e.v. pouch.

What I claim is that:

1) The e.v. pouch will be made of a thin latex material, thicker and more durable than a condom and not permeable to liquids or germs.

2) The e.v. pouch has an elongated shape with striated pleating which expands with defecation.

3) The e.v. pouch lays wide and flat at the bottom and expands as defecation occurs.

4) The bottom of the e.v. pouch contains a heavier strip of latex which re-inforces the strength during removal.
a. I further claims, the e.v. pouch will lay flat due to the thick latex at the bottom.
b. I further claim the heavier, wide strip of the e.v. pouch will facilitate the removal of the fecal material away from the body.

5) What I claim is
The neck of the E.V. Pouch leading from the mouth is narrower than the body but expands at the bottom of the pouch.

6) What I claim is
The outer adhesive mouth of the e.v. pouch fits flat against the outer skin surrounding the rectum.
a. I further claim it is attached to the e.v. pouch perpendicular.

7) What I claim is
The mouth of the pouch is lined with a non-irritating adhesive.
I further claim
a. The adhesive lined mouth is soft, flat, and pliable and will contour to the body.
b. The size of the adhesive will vary according to width and different sizes of the pouch.

8) What I claim is
The adhesive mouth is oval.

9) What I claim is
The adhesive mouth is round.

10) What I claim is
The adhesive mouth has a butterfly or four-sided club shape.

11) What I clam is
The adhesive mouth is rectangular.

12) What I claim is
The mouth portion when properly applied, opens up the e.v. pouch.

13) What I claim is
The e.v. pouch mouth fits the verticle (front-back) from the area directly above the rectum to an area directly below the rectum.

14) What I claim is
The adhesive mouth of the e.v. pouch fits around the rectum and creates a seal.

15) What I claim is
When the e.v. pouch is applied correctly the flat end of the pouch will lay flat against the bed or body.

16) What I claim is
The mouth of the e.v. pouch is flat, with the similarity of a thick piece of adhesive tape attached to a pouch.

17) What I claim is
The e.v. pouch has an elongated neck body with a shape similar to an open end isosceles triangle.

18) What I claim is
The rim comprising the adhesive mouth extends outward beyond the opening of the e.v. pouch.
